(19) United States
(12) Patent Application Publication
(10) Pub. No.: US 2012/0289433 A1
(43) Pub. Date: Nov. 15, 2012

(54) SOLID SURFACE RESERVOIRS

(75) Inventors: Zhuying Wang, Monmouth Junction, NJ (US); Weiming Jiang, Nanjing (CN); Jinyu Yang, Nanjing (CN); Lin Li, Nanjing (CN); Tao Bai, Nanjing (CN)

(73) Assignee: GENSCRIPT NANJING, Nanjing (CN)

(21) Appl. No.: 13/462,271

(22) Filed: May 2, 2012

Related U.S. Application Data
(60) Provisional application No. 61/481,482, filed on May 2, 2011.

(51) Int. Cl. C40B 60/14; C12M 1/42; G01N 33/53; G01N 1/44; C12M 3/00; C07K 1/04; C07H 21/00; B01J 19/00; B01J 19/12; C40B 50/14; C07K 2/00

(52) U.S. Cl. 506/30; 506/40; 435/283.1; 435/303.1; 435/7.1; 435/40.5; 435/7.92; 530/333; 536/25.3; 422/129; 422/109; 422/186.01; 435/289.1

(57) ABSTRACT

An apparatus for forming a surface reservoir to hold a sample for a desirable period of time is described. The apparatus contains a platform, a solid surface disposed onto the platform, and an assembly of a bottomless vessel mounted on the solid surface. Also described is an apparatus that forms an array of surface reservoirs on a solid surface when multiple bottomless vessels are used, which can be used for high throughput applications. The apparatus can be used in applications on a solid surface, such as immunohistochemistry (IHC), oligo synthesis, peptide synthesis, ELISA, DNA array, peptide array, protein array, antibody array, tissue array, cell culturing, etc.

### CROSS-REFERENCE TO RELATED APPLICATIONS

This application claims the benefit under 35 U.S.C. §119(e) of U.S. Provisional Application No. 61/481,482, filed May 2, 2011, the entire disclosure of which is hereby incorporated herein by reference.

### FIELD OF THE INVENTION

The present invention relates to an apparatus comprising a platform, a bottomless vessel and a solid surface for forming a temporary solid surface reservoir for a surface reaction, such as immunohistochemistry (IHC) reaction. The invention also relates to an apparatus comprising a platform, a plurality of bottomless vessels and a solid surface for forming an array of temporary solid surface reservoirs that can be used for culturing cells, as well as oligo synthesis, peptide synthesis, ELISA, DNA array, peptide array, protein array, antibody array, tissue array, etc. on the solid surface. The invention further relates to methods of assembling and using such apparatus.

### BACKGROUND OF THE INVENTION

Immunohistology or immunohistochemistry (IHC) refers to the process of detecting antigens in cells of a tissue section or cell sample based on the principle of antibodies binding specifically to antigens in biological tissues (Ramos-Vara, JA (2005). “Technical Aspects of Immunohistochemistry”, Vet Pathol 42 (4): 405-426). IHC has become a major tool to analyze the existence, localization and distribution of proteins of interest and is therefore widely used for diagnostic purposes, e.g., in the diagnosis of abnormal cells such as those found in cancerous tumors. Generally, during an IHC analysis, a tissue section or cell sample is fixed on the surface of a glass slide and then submitted to immunostaining with antigen specific antibodies.

One of the routine procedures of IHC process is to add the incubation solution over the tissue (or sample) fixed on a slide and cover it completely for binding reactions. However, there are a few problems associated with this technology. First, the slides need to be kept in a humidified chamber to prevent drying up of the tissue or sample during the process because the incubation solution will evaporate into the open air. Second, the incubation solution could spread beyond the sample area that needs to be covered because there is no proper container to hold the incubation solution within specific boundary, which results in either drying up of the tissue or wasting of the reagents, especially valuable antibodies. Finally, only one tissue or sample is examined on each slide due to the concern of possible contamination caused by the spreading around of the incubation solutions, which limits the throughput of this powerful assay method.

Therefore, there exists a need for a device that can temporarily physically separate a region containing the tissue or sample from other area and hold the incubation solution within its boundary. After the incubation is complete, the device can be easily removed and the slide is then further processed, such as being scanned or observed under a microscope. In particular, there exists a need for an IHC apparatus that temporarily forms one or more surface reservoirs on the glass slide and meet multiple goals, e.g., 1) to seal the incubation reaction to prevent solution evaporation; 2) to hold the incubation solution to prevent the waste of valuable reagents; and 3) to allow multiple IHC experiments to be performed in parallel on the same slides to make the assays high throughput.

The need for an apparatus that can form temporary surface reservoirs is not limited to IHC experiments. Such need extends more generally to other applications, such as peptide array synthesis, oligonucleotide array synthesis, etc. Additional applications of such apparatus also include, for example, using the multiple reservoirs as bioreactors for use in culturing cells. After separate reaction or culturing in the temporary reservoirs, the peptides, the oligonucleotides, the cells, etc. can be either processed or tested under the same or different conditions, which allows multiple experiments to be performed in parallel at high throughput.

### BRIEF SUMMARY OF THE INVENTION

In one general aspect, the present invention relates to a solid surface apparatus for forming a surface reservoir to hold a sample for a desirable period of time, comprising: a platform, a solid surface disposed onto the platform, and an assembly of a bottomless vessel mounted on the solid surface, wherein the assembly forms contact with the solid surface at an open end of the vessel to thereby form the surface reservoir for holding the sample for the desirable period of time.

In another general aspect, the present invention relates to an apparatus for forming a surface reservoir to hold a sample for a desirable period of time, comprising:

(a) a magnetic platform comprising a permanent magnet or an electromagnet;

(b) a solid surface disposed onto the magnetic platform; and

(c) an assembly of a bottomless vessel mounted on the solid surface to form a contact with the solid surface at an open end of the vessel,

wherein the assembly comprises a ferromagnetic or ferrimagnetic material, and the magnetic force between the platform and the assembly adheres the vessel onto the solid surface to thereby form the surface reservoir for holding the sample for the desirable period of time.

Another general aspect of the invention relates to an apparatus for forming a surface reservoir to hold a sample for a desirable period of time, comprising:

(a) a magnetic platform comprising a permanent magnet or an electromagnet;

(b) a solid surface disposed onto the magnetic platform;

(c) an assembly of a bottomless vessel mounted on the solid surface to form a contact with the solid surface at an open end of the vessel, wherein the assembly comprises a flange, and a first surface of the flange forms the contact with the solid surface at the open end of the vessel; and

(d) an O-ring comprising a ferromagnetic or ferrimagnetic material placed on a second surface of the flange opposing to the first surface of the flange,

wherein the magnetic force between the platform and the O-ring presses the flange against the solid surface and adheres the vessel onto the solid surface to thereby form the surface reservoir for holding the sample for the desirable period of time.

Other aspects of the invention relates to methods of conducting one or more reactions on a solid surface, comprising:

(a) forming a surface reservoir according to an embodiment of the invention;

(b) conducting the one or more reactions within the surface reservoir; and

(c) disassembling the surface reservoir after the one or more reactions are completed.

An aspect of the invention also relates to a method of forming a surface reservoir for holding a sample for a desirable period of time, comprising:

(a) providing a magnetic platform comprising a permanent magnet or an electromagnet;

(b) placing a solid surface onto the magnetic platform; and

(c) mounting an assembly of a bottomless vessel on the solid surface to form a contact with the solid surface at an open end of the vessel,

wherein the assembly comprises a ferromagnetic or ferrimagnetic material, and the magnetic force between the platform and the assembly adheres the vessel onto the solid surface to thereby form the surface reservoir for holding the sample for the desirable period of time.

Another aspect of the invention relates to a method of forming a surface reservoir for holding a sample for a desirable period of time, comprising:

(a) providing a magnetic platform comprising a permanent magnet or an electromagnet;

(b) placing a solid surface onto the magnetic platform;

(c) mounting an assembly of a bottomless vessel on the solid surface to form a contact with the solid surface at an open end of the vessel, wherein the assembly comprises a flange, and a first surface of the flange forms the contact with the solid surface at the open end of the vessel; and

(d) placing an O-ring comprising a ferromagnetic or ferrimagnetic material on a second surface of the flange opposing to the first surface of the flange,

wherein the magnetic force between the platform and the O-ring presses the flange against the solid surface and adheres the vessel onto the solid surface to thereby form the surface reservoir for holding the sample for the desirable period of time.

The details of one or more embodiments of the disclosure are set forth in the accompanying drawings and the description below. Other features, objects, and advantages will be apparent from the description and drawings, and from the claims.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited by the drawings.

FIG. 1 is a graphic illustration of an apparatus according to an embodiment of the invention, the apparatus has a platform, which contains a permanent magnet or an electromagnet, a solid surface, and an assembly of bottomless vessel that contains a ferromagnetic (or ferrimagnetic) material, the magnetic force between the platform and the bottomless vessel tightly squeezes or adheres the bottomless vessel to the solid surface to form a surface reservoir, which can be easily disassembled by removing the vessel, or by switching off the electricity for the electromagnet;

FIG. 2 is a graphic illustration of an apparatus according to another embodiment of the invention, the apparatus has a platform, which contains a permanent magnet or an electromagnet, a solid surface, a nonmagnetic bottomless vessel, and an O-ring (not shown) that contains a ferromagnetic (or ferrimagnetic) material, the magnetic force between the platform and the O-ring tightly attaches the bottomless vessel to the solid surface to form a surface reservoir, which can be easily disassembled by removing the vessel or O-ring, or by switching off the electricity for the electromagnet;

FIG. 3 is a photo picture of an assembly comprising multiple bottomless vessels that can be used in an apparatus according to an embodiment of the invention; and

FIG. 4 is a photo picture of apoptotic cells detected from mouse liver tissue paraffin-embedded on a glass slide using an IHC apparatus according to an embodiment of the invention, i.e., a TUNEL™ kit (GenScript, L00290).

### DETAILED DESCRIPTION OF THE INVENTION

Unless defined otherwise, all technical and scientific terms used herein have the same meaning as is commonly understood by one of skill in the art to which this invention belongs. All publications and patents referred to herein are incorporated by reference.

As used herein, the terms “immunohistochemistry”, “immunohistology”, “immunostaining”, a “platform”, a “solid surface”, a “bottomless vessel”, a “magnet”, a “polymer”, “plastics”, “ferromagnetic”, “ferrimagnetic”, “polynucleotide”, “peptide”, “antibody” and “protein” are to be taken in its broadest context. The terms “immunohistochemistry”, “immunohistology” and “immunostaining” are used interchangeably. “Ferromagnetic” and “ferrimagnetic” are used interchangeably.

Embodiments of the current invention relate to a variety of apparatus for chemical, biological or biochemical surface reactions and associated devices, as well as methods for using and manufacturing the apparatus. The apparatus comprises a platform, a bottomless vessel and a solid surface that can be used for surface reaction and other purposes.

For example, a magnetic force between the platform, which contains a permanent magnet or an electromagnet, and the bottomless vessel, which contains ferromagnetic (or ferrimagnetic) materials, tightly squeezes or adheres the bottomless vessel to the solid surface to form a surface reservoir, which can be easily disassembled by removing the ferromagnetic (or ferrimagnetic) material containing vessel or by switching off the electricity. The bottomless vessel can be covered by a removable cap or lid or an attached cap or lid to seal the reservoir.

Alternatively, a ferromagnetic (or ferrimagnetic) O-ring can be used to tightly attach a nonmagnetic bottomless vessel to the solid surface to form a surface reservoir for any desirable period of time.

Embodiments of the invention relate to an apparatus, such as an immunohistochemistry (IHC) apparatus. The bottomless vessel and the solid surface, such as a slide, form a temporary surface reservoir to hold the incubation solution within a boundary. The reservoir is covered by a removable cap or an attached cap to seal the reservoir for a long incubation time such as hours or even overnight. The entire IHC apparatus can be kept in an incubator for incubation at a specific temperature. After the immunostaining process, the vessel can be removed and the slide is then subject to further analysis such as scanning or observation under a microscope.

Other embodiments of the invention relate to an apparatus, such as an IHC apparatus, that allows multiple IHC experiments to be performed in parallel at high throughput. An assembly comprising a plurality of bottomless vessels is used to set up a plurality temporary surface reservoirs. The apparatus allows multiple experiments, such as IHC analysis, to be performed in parallel, which increases the throughput of the assay significantly. Instead of processing one tissue (or cell sample) at a time, multiple tissues (or cell samples) can be processed at the same time.

Embodiments of the invention relate to an apparatus that forms an array of small-scale surface reservoirs that can be used for testing and/or optimizing any type of chemical, biochemical or biological reactions that can occur on a solid surface such as a glass slide. These reactions include, but are not limited to, ELISA, peptide synthesis, oligonucleotide synthesis, analysis on DNA array, peptide array, protein array, antibody array or tissue array, etc.

Embodiments of the invention also relate to an apparatus that forms an array of small-scale surface reservoirs that can be used as bioreactors for culturing cells, cell-based array analysis, etc.

According to an embodiment of the invention, an apparatus for forming a surface reservoir to hold a sample for a desirable period of time, comprises:

(a) a magnetic platform comprising a permanent magnet or an electromagnet;

(b) a solid surface disposed onto the magnetic platform; and

(c) an assembly of a bottomless vessel mounted on the solid surface to form a contact with the solid surface at an open end of the vessel,

wherein the assembly comprises a ferromagnetic or ferrimagnetic material, and the magnetic force between the platform and the assembly adheres the vessel onto the solid surface to thereby form the surface reservoir for holding the sample for the desirable period of time.

In a preferred embodiment, the apparatus is an immunohistochemistry (IHC) apparatus.

In another preferred embodiment, the sample is a liquid sample.

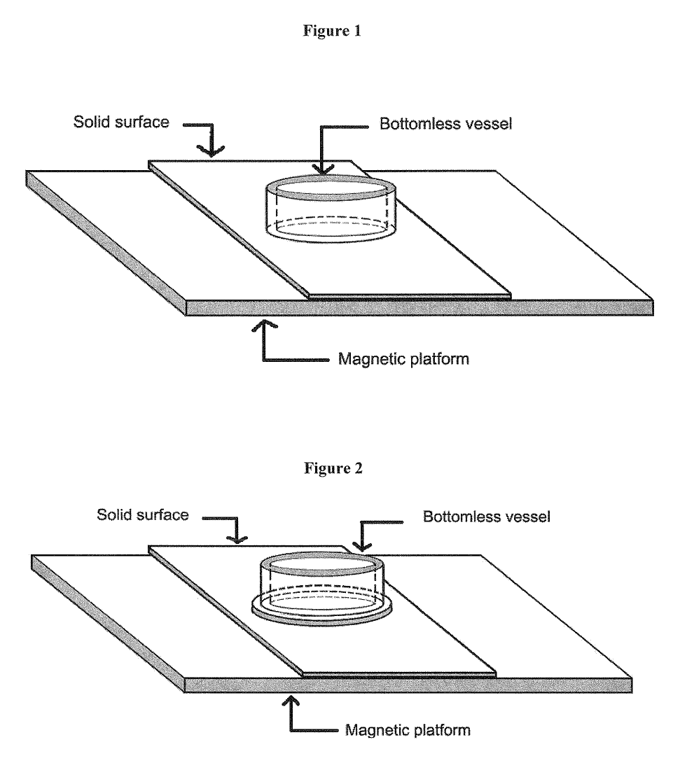

An embodiment of such an apparatus is depicted in FIG. 1. A magnetic platform, an assembly of a bottomless vessel and a solid surface are assembled together as shown in the figure. The bottomless vessel comprises a ferromagnetic (or ferrimagnetic) material. When mounted on the solid surface placed on top of the magnetic platform, the magnetic force between the magnetic platform and the bottomless vessel tightly squeezes or adheres the bottomless vessel to the solid surface. A temporary surface reservoir is then formed by the wall of the bottomless vessel and the solid surface.

In one embodiment of the invention, the assembly comprises a plurality of bottomless vessels. The assembly comprises a ferromagnetic or ferrimagnetic material in at least one of walls of the plurality of bottomless vessels and space connecting the plurality of bottomless vessels. The magnetic force between the platform and the assembly tightly squeezes or adheres the plurality of the bottomless vessels onto the solid surface to form an array of surface reservoirs for holding one or more samples, preferably liquid samples.

Another aspect of the invention relates to an apparatus comprising a platform, a solid surface, a bottomless vessel and an O-ring that can be used for chemical, biological or biochemical surface reaction.

According to an embodiment of the invention, an apparatus for forming a surface reservoir to hold a sample for a desirable period of time, comprises:

(a) a magnetic platform comprising a permanent magnet or an electromagnet;

(b) a solid surface disposed onto the magnetic platform;

(c) an assembly of a bottomless vessel mounted on the solid surface to form a contact with the solid surface at an open end of the vessel, wherein the assembly comprises a flange, and a first surface of the flange forms the contact with the solid surface at the open end of the vessel; and

(d) an O-ring comprising a ferromagnetic or ferrimagnetic material placed on a second surface of the flange opposing to the first surface of the flange,

wherein the magnetic force between the platform and the O-ring presses the flange against the solid surface and adheres the vessel onto the solid surface to thereby form the surface reservoir for holding the sample for the desirable period of time.

An embodiment of such an apparatus is depicted in FIG. 2. A magnetic platform, a bottomless vessel, a solid surface and an O-ring (not shown) are assembled together as shown in the figure. The O-ring contains a ferromagnetic or ferrimagnetic material. In FIG. 2, the bottomless vessel has a flange at the lower open end of the vessel. The lower surface of the flange is mounted on the solid surface. The O-ring (not shown) is placed on the upper surface of the flange outside of the bottomless vessel. The magnetic force between the magnetic platform and the O-ring tightly squeezes the bottomless vessel to the solid surface placed on top of the platform to form a temporary surface reservoir having the wall of the bottomless vessel and the bottom of the solid surface.

In one embodiment of the invention, the assembly comprises a plurality of the bottomless vessels. The assembly has a flange. A first surface of the flange forms a contact with the solid surface at an open end of each of the plurality of the bottomless vessels. A ferromagnetic or ferrimagnetic O-ring is placed on a second surface of the flange opposing to the first surface of the flange. The magnetic force between the magnetic platform and the O-ring tightly squeezes or adheres the assembly, thus the plurality of the bottomless vessels, onto the second surface of the solid surface to form an array of surface reservoirs for holding a plurality of samples.

According to embodiments of the present invention, the platform comprises one or more permanent magnets, or one or more electromagnets whose magnetic fields are produced by the flow of electric current.

According to embodiments of the present invention, the solid surface, i.e., the surface that serves as the bottom of the temporary surface reservoir, can be made of any suitable material in view of the present disclosure, such as glass, silicon, membrane plastics, etc. For example, the solid surface can be a glass slide or a silicon wafer on which tissue section is paraffin-embedded or cells are fixed.

Preferably, the solid surface is treated, e.g., physically, chemically, biologically or biochemically. In one embodiment of the present invention, the solid surface is physically treated to increase the surface area. In another embodiment of the present invention, the solid surface is chemically treated to support peptide synthesis or oligonucleotide synthesis. In yet another embodiment of the present invention, the solid surface is chemically or biologically treated for culturing cells or biochemically treated for protein or antibody binding. In a particular embodiment of the invention, the solid surface is treated to allow a biological sample, such as a tissue section, cell sample, polypeptide or nucleic acid sample, to be fixed thereon.

According to embodiments of the present invention, the bottomless vessel can have any shape suitable for the purpose of the surface reservoir, such as round, square-shaped or even rectangular-shaped.

As used herein, the term “O-ring” encompasses any shape of the ring suitable to press the assembly of a bottomless vessel against the solid surface and adheres the vessel onto the solid surface to form a surface reservoir for holding a sample, preferably, a liquid sample. The O-ring can have or adopt any shape according to the outside shape of the assembly.

According to embodiments of the present invention, the bottomless vessel or the O-ring comprises at least one ferromagnetic or ferrimagnetic material selected from the group consisting of cobalt, iron, nickel, alloys made of cobalt, iron and nickel, metal oxides, sulfides, oxyhydroxides, and any other suitable ferromagnetic or ferrimagnetic material.

According to other embodiments of the present invention, the bottomless vessel is made of or encapsulated in plastics that is selected from the group consisting of an elastomer, a rubber, silicone, neoprene, nylon, PVC, polystyrene, polyethylene, polypropylene, polyacrylonitrile, PVB, and any other suitable material.

According to an embodiment of the present invention, the platform is mounted on or attached to a base that can also comprise a switch of an electricity power and connection to the electricity power.

The apparatus according to an embodiment of the present invention can be enclosed within an appropriate housing which comprises at least one of an electricity power access for the electromagnet, a vacuum pump and one or more conduits for adding or removing at least a portion of the sample, such as the incubation or wash solutions, a temperature controller for maintaining the incubation at a preferred temperature, a moisture controller, etc. For example, an apparatus according to an embodiment of the present invention can be enclosed within an appropriate housing which comprises robotics for automatic addition or removal of the incubation solutions, a temperature controlling system for incubation, other functions and software and computers for controlling such procedures.

In one embodiment of the invention, the apparatus comprises a plurality of the bottomless vessels, each of the vessels independently can be round, square-shaped or even rectangular-shaped, thus forming a plurality of the surface reservoirs. Such apparatus can be used for high throughput purposes.

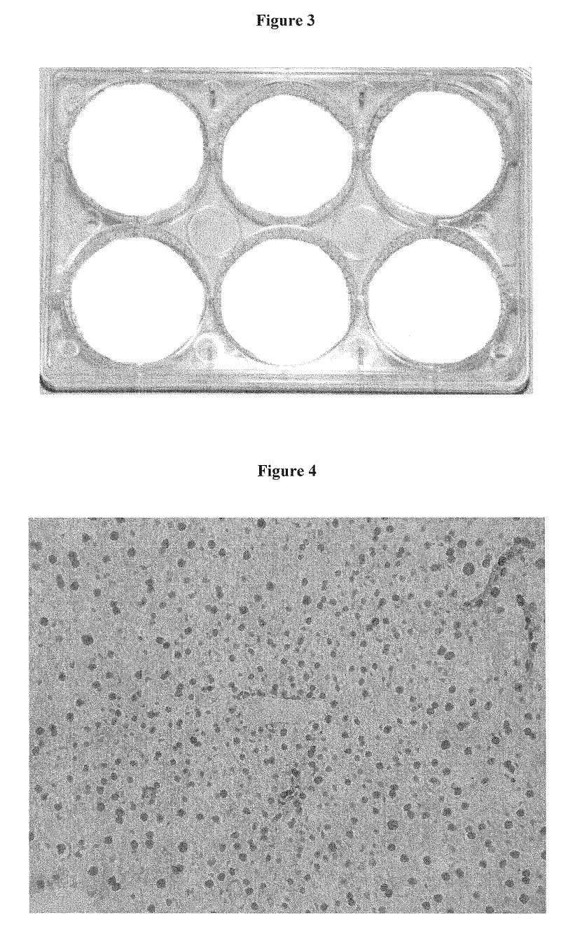

In a preferred embodiment, the plurality of bottomless vessels are fabricated in a single assembly, such as the bottomless 6-vessel (6-well) assembly illustrated in FIG. 3, in an apparatus according to an embodiment of the present invention. An assembly containing more bottomless vessels at smaller scales, such as an assembly having 48-vessels, 96-vessels, 192-vessels, or even 384-vessels, can be used to form an array of small-scale surface reservoirs, such as 48-wells, 96-wells, 192-wells, or even 384-wells, for high throughput purposes.

Unlike spot arrays, these small-scale surface reservoirs formed by an apparatus according to embodiments of the present invention are physically separated from one another, thus preventing possible contamination thereof. Many chemical, biochemical or biological reactions that occur on solid surfaces can be performed in the array of surface reservoirs. These reactions include, but are not limited to, ELISA, peptide synthesis, oligonucleotide synthesis, DNA array, peptide array, protein array, antibody array, tissue array, etc.

The array of surface reservoirs can be covered by a lid or multiple removable caps or attached caps to seal the reservoirs for a long incubation time such as hours or even overnight.

A further aspect of the present invention relates to a method of forming a surface reservoir for holding a sample, preferably a liquid sample. The method comprises: (a) providing a magnetic platform comprising a permanent magnet or an electromagnet; (b) placing a solid surface onto the magnetic platform; and (c) mounting an assembly of a bottomless vessel comprising a ferromagnetic or ferrimagnetic material on the solid surface, wherein the assembly forms a contact with the solid surface at an open end of the vessel, and the magnetic force between the platform and the assembly adheres the vessel onto the solid surface to thereby form the surface reservoir for holding the sample.

Embodiments of the invention further relate to a method of forming a surface reservoir for holding a sample, preferably a liquid sample, comprising: (a) providing a magnetic platform comprising a permanent magnet or an electromagnet; (b) placing a solid surface onto the magnetic platform; (c) mounting an assembly of a bottomless vessel on the solid surface, wherein the assembly comprises a flange, and a first surface of the flange forms a contact with the solid surface at an open end of the vessel; and (d) placing an O-ring comprising a ferromagnetic or ferrimagnetic material on a second surface of the flange opposing to the first surface of the flange, wherein the magnetic force between the platform and the O-ring presses the flange against the solid surface and adheres the vessel onto the solid surface to thereby form the surface reservoir for holding the sample, preferably, the liquid sample.

A further aspect of the present invention relates to a method of conducting one or more reactions on a solid surface. The method comprises: (a) forming one or more surface reservoirs according to an embodiment of the present invention; (b) conducting the one or more reactions within the surface reservoirs; and (c) disassembling the surface reservoirs after the one or more reactions are completed.

Reactions that can be conducted using the present invention, include, but are not limited to, steps or procedures involved in IHC, cell culturing, oligo synthesis, peptide synthesis, ELISA, DNA array, peptide array, protein array, antibody array, tissue array, etc.

Methods according to an embodiment of the present invention can further comprise preparing the solid surface for a reaction, preferably before assembling the solid surface to form the surface reservoirs.

Methods according to an embodiment of the present invention can further comprise processing and/or analyzing the solid surface after the one or more reaction in the surface reservoir are completed, preferably after disassembling the surface reservoirs after the one or more reactions are completed.

Embodiments of the invention also relate to a flexible apparatus system that can be assembled at any stage during a chemical or biological process. Furthermore, the invention also provides a flexible apparatus system that can be disassembled at any stage during a chemical or biological process.

For example, to perform a high throughput IHC, more than one tissue samples can be fixed on a glass. No surface reservoir is needed at this tissue fixing stage. However, when performing immunostaining reactions, an apparatus having multiple vessels is preferred to form multiple temporary surface reservoirs to prevent contamination and reagent wasting, especially when several different antibodies are used. At the last stage of IHC, when the glass slide is to be examined under a microscope, the temporary reservoirs can be easily disassembled by removing the slides from the magnetic platform or by simply switching off the electric power of the electromagnet.

In another embodiment of the invention, the apparatus can be used as a peptide array for antibody epitope mapping. For example, a glass slide surface is first chemically processed to be ready for peptide synthesis. Then, an array of temporary small-scale surface reservoirs is set up on the glass slide surface according to several embodiments of the invention. Subsequently, an array of peptides is synthesized in the small-scale surface reservoirs using methods known in the art in view of the present disclosure. The temporary surface reservoirs are then disassembled by removing the slides from the magnetic platform or by simply switching off the electric power of the electromagnet. The slide with the array of peptides can then be processed or analyzed, e.g., by immunostaining to determine the epitope sequence.

Still another example to use the invention is a cell array, where an apparatus according to an embodiment of the invention is used to form an array of surface reservoirs that serve as bioreactors for culturing cells on a surface. First, a glass slide surface is processed to be ready for culturing cells. Second, an array of temporary small-scale surface reservoirs is set up according to several embodiments of the invention. Third, an array of cells is seeded and cultured in the small-scale surface reservoirs. Finally, the temporary reservoirs are disassembled by removing the slides from the magnetic platform or by simply switching off the electric power of the electromagnet, and the slide with an array of cells can now be processed or analyzed, e.g., by immunostaining or cell-based assays.

In view of the present disclosure, the magnetic platform can comprise either a permanent magnet or an electromagnet whose magnetic field is produced by the flow of electric current, both are known to those skilled in the art. The magnetic field of an electromagnet can be simply controlled by electric power and therefore an electromagnet is a preferred option for forming a temporary reservoir according to embodiments of the invention. The magnetic platform can also comprise more than one permanent magnet or electromagnet for stronger magnetic force or a bigger area.

The platform can be made to support one slide or a few glass slides as used in immunohistology, or it can be made to support as many glass slides as necessary.

In view of the present disclosure, the assembly of the bottomless vessel, or at least the flange of the assembly, is preferably made of slightly soft materials such as plastics so as to form a temporary surface reservoir without leakage when the end surface of the vessel or the end surface of the flange touches the solid surface. One of the preferred plastics is an elastomer.

The end surface of the vessel or the flange that is to be placed immediately adjacent to the solid surface is preferably hydrophobic so as to reduce the absorption of aqueous incubation solution, to therefore prevent the wasting of incubation solution. The end surface of the vessel or the flange can also be made to be hydrophilic so as to reduce the absorption of hydrophobic incubation solution to therefore prevent the wasting of incubation solution.

There are several ways to incorporate ferromagnetic (or ferrimagnetic) materials into an assembly of bottomless vessel to produce the magnetic force between the magnetic platform and the vessel. For example, the assembly can be made of one or more magnetic polymers. The assembly can also be made of plastics with magnetic nanoparticles incorporated. In addition, the bottomless vessel can be made of ferromagnetic (or ferrimagnetic) materials such as cobalt or iron which is then encapsulated in plastics that can be selected from, but not limited to, an elastomer, a rubber, silicone, neoprene, nylon, PVC, polystyrene, polyethylene, polypropylene, polyacrylonitrile, PVB, etc. Other methods can also be used to incorporate ferromagnetic or ferrimagnetic materials into the assembly of bottomless vessel in view of the present disclosure.

In the above-mentioned embodiments, those skilled in the art will understand that many materials are ferromagnetic (or ferrimagnetic) materials. These materials include, but not limited to, cobalt, iron, nickel, alloys made of cobalt, iron and nickel, metal oxides, sulfides, oxyhydroxides, etc. These materials can be made in the form of nano particles and incorporated into plastics. In view of the present disclosure, any of the ferromagnetic (or ferrimagnetic) materials in suitable forms can be incorporated into the assembly of bottomless vessels as depicted in FIG. 1 or the O-rings as depicted in FIG. 2 using methods known in the art.

In view of the present disclosure, those of ordinary skill in the art would readily appreciate that the bottomless vessel or the bottomless well in an array assembly can be made to be cylindrical shape, a square box shape, a triangle shape, a round shape, polygonal shapes, mixtures thereof, or any other shapes. Accordingly, a round shaped ring, a square shaped ring, or any other shaped ring can be used as the O-ring to hold the vessel tightly to the solid surface if the vessel is made of materials which do not contain ferromagnetic (or ferrimagnetic) materials.

In view of the present disclosure, it is readily appreciated that many materials can be used as the solid surfaces for surface reactions. These solid surfaces include, but not limited to, glass, silicon, membrane or plastics, etc. These solid surfaces can be processed chemically to include one or more functional groups such as amino group, carboxylic acid group, hydroxyl group or thiol group, etc. They can be also processed biochemically to enhance protein binding or to support cell growth. Furthermore, they can be even treated physically to increase the surface area.

According to embodiments of the present invention, the apparatus can comprise one or more additional components. For example, the magnetic platform of the invention can be mounted on or attached to a base which can also comprise a switch of and connection to electricity power. The magnetic platform can also comprise one or more holders for solid surfaces such as glass slides. The magnetic platform, the solid surfaces, and the bottomless vessels with or without the O-ring of the invention can also be enclosed within appropriate housing. The housing can comprise electricity power access for the electromagnet, a vacuum pump and related tubes for removing incubation or wash solutions, and a temperature controller for maintaining the incubation at a preferred temperature. Furthermore, the magnetic platform, the solid surfaces, and the bottomless vessels with or without the O-ring of the invention can be enclosed within appropriate housing which encloses robotics for automatic addition or removal of the incubation solutions, temperature controlling system for incubation, and software and computers for controlling such procedures.

Various embodiments of the invention have now been described. It is to be noted, however, that this description of these specific embodiments is merely illustrative of the principles underlying the inventive concept. It is therefore contemplated that various modifications of the disclosed embodiments will, without departing from the spirit and scope of the invention, be apparent to persons skilled in the art.

The following specific example of the methods of the invention is further illustrative of the nature of the invention, it needs to be understood that the invention is not limited thereto.

### EXAMPLES

Mouse liver tissue paraffin-embedded on a slide was immunostained by terminal deoxynucleotidyl transferase (TdT)-mediated dUTP nick end labeling (TUNEL™) kit (GenScript, L00290) for the detection of apoptotic cells. After the slide was dewaxed in xylene, a temporary surface reservoir was assembled by placing the slide onto a permanent magnet, and then placing a bottomless iron vessel encapsulated in silicone onto the slide with the bottomless vessel enclosing the tissue.

All of the following steps were performed in the temporary surface reservoir: (1) rehydration of the tissue in graded ethanol series to water; (2) permeabilization of tissue with proteinase K; (3) TUNEL™ labeling with TdT enzyme mix; (4) detection with SA-HRP; and (5) staining with DAB mix.

After the staining step (5) was completed, the temporary surface reservoir was disassembled by removing the bottomless vessel from the plate and the glass slide was examined under a microscope. A picture of the detected apoptotic cells was taken from the microscope and was shown in FIG. 4.

I/We claim:

1. A solid surface apparatus for forming a surface reservoir to hold a sample for a desirable period of time, comprising: a platform, a solid surface disposed onto the platform, and an assembly of a bottomless vessel mounted on the solid surface, wherein the assembly forms contact with the solid surface at an open end of the vessel to thereby form the surface reservoir for holding the sample for the desirable period of time.

2. A method of conducting one or more reactions on a solid surface, comprising:
(a) forming the surface reservoir on the solid surface according to claim 1;
(b) conducting the one or more reactions within the surface reservoir; and
(c) disassembling the surface reservoir after the one or more reactions are completed.

3. An apparatus for forming a surface reservoir to hold a sample for a desirable period of time, comprising:
(a) a magnetic platform comprising a permanent magnet or an electromagnet;
(b) a solid surface disposed onto the magnetic platform; and
(c) an assembly of a bottomless vessel mounted on the solid surface to form a contact with the solid surface at an open end of the vessel,
wherein the assembly comprises a ferromagnetic or ferrimagnetic material, and the magnetic force between the platform and the assembly adheres the vessel onto the solid surface to thereby form the surface reservoir for holding the sample for the desirable period of time.

4. The apparatus of claim 3, wherein the platform comprises one or more electromagnets.

5. The apparatus of claim 3, wherein the solid surface is physically, chemically, biologically or biochemically treated.

6. The apparatus of claim 3, wherein the apparatus further comprises a cover or lid for the surface reservoir.

7. The apparatus of claim 3, wherein the assembly comprises a plurality of bottomless vessels.

8. The apparatus of claim 3, being enclosed within a housing that comprises at least one of an electricity power access, a conduit for adding or removing at least a portion of the sample, a temperature controller for controlling the temperature of the sample, and a moisture controller.

9. The apparatus of claim 3 being an immunohistochemistry (IHC) apparatus.

10. A method of conducting one or more reactions on a solid surface, comprising:
(a) forming a surface reservoir according to claim 3;
(b) conducting the one or more reactions within the surface reservoir; and
(c) disassembling the surface reservoir after the one or more reactions are completed.

11. The method of claim 10, further comprising treating the solid surface for the one or more reactions prior to forming the surface reservoir.

12. An apparatus for forming a surface reservoir to hold a sample for a desirable period of time, comprising:
(a) a magnetic platform comprising a permanent magnet or an electromagnet;
(b) a solid surface disposed onto the magnetic platform;
(c) an assembly of a bottomless vessel mounted on the solid surface to form a contact with the solid surface at an open end of the vessel, wherein the assembly comprises a flange, and a first surface of the flange forms the contact with the solid surface at the open end of the vessel; and
(d) an O-ring comprising a ferromagnetic or ferrimagnetic material placed on a second surface of the flange opposing to the first surface of the flange,
wherein the magnetic force between the platform and the O-ring presses the flange against the solid surface and adheres the vessel onto the solid surface to thereby form the surface reservoir for holding the sample for the desirable period of time.

13. The apparatus of claim 12, wherein the platform comprises one or more electromagnets.

14. The apparatus of claim 12, wherein the solid surface is physically, chemically, biologically or biochemically treated.

15. The apparatus of claim 14, wherein the apparatus further comprises a cover or lid for the surface reservoir.

16. The apparatus of claim 12, wherein the assembly comprises a plurality of bottomless vessels.

17. The apparatus of claim 12, being enclosed within a housing that comprises at least one of an electricity power access, a conduit for adding or removing at least a portion of the sample, a temperature controller for controlling the temperature of the sample, and a moisture controller.

18. The apparatus of claim 12 being an immunohistochemistry (IHC) apparatus.

19. A method of conducting one or more reactions on a solid surface, comprising:
(a) forming a surface reservoir according to claim 12;
(b) conducting the one or more reactions within the surface reservoir; and
(c) disassembling the surface reservoir after the one or more reactions are completed.

20. The method of claim 19, further comprising treating the solid surface for the one or more reactions prior to forming the surface reservoir.

21. A method of forming a surface reservoir for holding a sample for a desirable period of time, comprising:
(a) providing a magnetic platform comprising a permanent magnet or an electromagnet;
(b) placing a solid surface onto the magnetic platform; and
(c) mounting an assembly of a bottomless vessel on the solid surface to form a contact with the solid surface at an open end of the vessel,
wherein the assembly comprises a ferromagnetic or ferrimagnetic material, and the magnetic force between the platform and the assembly adheres the vessel onto the solid surface to thereby form the surface reservoir for holding the sample for the desirable period of time.

22. A method of forming a surface reservoir for holding a sample for a desirable period of time, comprising:
(a) providing a magnetic platform comprising a permanent magnet or an electromagnet;
(b) placing a solid surface onto the magnetic platform;
(c) mounting an assembly of a bottomless vessel on the solid surface to form a contact with the solid surface at an open end of the vessel, wherein the assembly comprises a flange, and a first surface of the flange forms the contact with the solid surface at the open end of the vessel; and
(d) placing an O-ring comprising a ferromagnetic or ferrimagnetic material on a second surface of the flange opposing to the first surface of the flange,
wherein the magnetic force between the platform and the O-ring presses the flange against the solid surface and adheres the vessel onto the solid surface to thereby form the surface reservoir for holding the sample for the desirable period of time.
